(19) United States
(12) Patent Application Publication
(10) Pub. No.: US 2024/0181122 A1
(43) Pub. Date: Jun. 6, 2024

(54) POROUS SHEET PRODUCT AND METHODS FOR MAKING AND USING THE SAME

(71) Applicant: William Winchin Yen, Arlington, MA (US)

(72) Inventor: William Winchin Yen, Arlington, MA (US)

(21) Appl. No.: 18/287,606

(22) PCT Filed: Apr. 19, 2022
(86) PCT No.: PCT/US2022/025414

§ 371 Date: Oct. 19, 2023

Related U.S. Application Data
(60) Provisional application No. 63/177,119, filed on Apr. 20, 2021.
(60) Provisional application No. 63/301,224, filed on Jan. 20, 2022.

(51) Int. Cl. A61L 15/60; A61F 13/15; A61F 13/53; A61F 13/534; A61F 13/84; A61L 15/22; B01J 20/26; B01J 20/28; B01J 20/30

(52) U.S. Cl. CPC A61L 15/60; A61F 13/8405; A61L 15/22; B01J 20/261; B01J 20/28004; B01J 20/28026; B01J 20/28035; B01J 20/28045; B01J 20/3007; B01J 20/3021; B01J 20/3042; B01J 20/3078; A61F 2013/15373; A61F 2013/15967; A61F 2013/530569; A61F 2013/530875; A61F 2013/53445

(57) ABSTRACT

Porous sheet product and methods of making and using the same. In one embodiment, the porous sheet product may be made by a process that includes providing an extrusion mixture, the extrusion mixture including a polymeric binder, a swelling agent, a compatibilizing agent, and a superabsorbent polymer, the superabsorbent polymer being in particle form. The superabsorbent polymer may be wet-milled such that its particle size before and after milling is reduced by a ratio of at least 5:1. The extrusion mixture may be melt-extruded to form a sheet material in film form. At least a portion of the swelling and compatibilizing agents may be removed. The porous sheet product is preferably liquid permeable and may be used, for example, as a battery separator, as a food packaging material, as a diffusion barrier and disposable garments.

### CROSS-REFERENCE TO RELATED APPLICATIONS

The present application claims the benefit under 35 U.S.C. 119(e) of U.S. Provisional Patent Application No. 63/177,119, inventor William Winchin Yen, filed Apr. 20, 2021, and of U.S. Provisional Patent Application No. 63/301,224, inventor William Winchin Yen, filed Jan. 20, 2022, the disclosures of which are incorporated herein by reference in their entireties.

### BACKGROUND OF THE INVENTION

The present invention relates generally to porous sheet products and relates more particularly to a novel porous sheet product and to methods of making and using the same. Porous sheet products are well-known and commonly used articles found in items as diverse as, for example, storage batteries, food packaging materials, and ultrafiltration devices. For example, in storage batteries, porous sheet products are commonly used as battery separators. Typically, a storage battery includes at least one pair of electrodes of opposite polarity and, in many cases, includes a series of electrode pairs of alternating polarity. The current flow between the electrodes of each pair is maintained by an electrolyte. Depending on the nature of the battery system, the electrolyte may be acidic, alkaline, or substantially organic aprotic. For example, in alkaline storage batteries, which include, but are not limited to, primary, secondary, nickel, zinc and silver cells, the electrolyte is generally an aqueous solution of potassium hydroxide. By contrast, in lead acid batteries, the electrolyte is typically a sulfuric acid solution, and, in lithium rechargeables, the electrolyte is typically a lithium salt solution in an aprotic organic solvent or solvent blend.

A battery separator is typically provided in a storage battery between adjacent electrodes of opposite polarity to prevent direct contact between the oppositely charged electrode plates since such direct contact would result in a short circuit of the battery. In general, it is highly desirable for the separator to possess one or more of the following qualities: (i) to be thin and lightweight to aid in providing a battery of high energy density and specific energy; (ii) to have a structure that inhibits dendrite formation between the electrode plates; (iii) to have the ability to enhance the uptake of the electrolytic composition over the electrode plates and, in so doing, to promote a substantially uniform distribution of the electrolytic composition over the electrode plates (an effect generally referred to as wicking); (iv) to provide the property of freely permitting electrolytic conduction; and (v) to have a dimensionally stable structure even during thermal excursions (internal or external heating). It is further highly desirable for the separator to be made in an economical and environmentally safe manner while being substantially free of defects, such as pinholes and the like.

One known type of separator comprises a porous sheet product that is formed by extruding a composition that includes a polyolefin and a solvent and, thereafter, removing the solvent to produce a sheet with a porous structure. An example of such a separator is disclosed in U.S. Patent Application Publication No. US 2013/0029126 A1, inventor Yen, which was published Jan. 31, 2013, and which is incorporated herein by reference. In particular, in the aforementioned publication, there is disclosed a sheet product suitable for use as a battery separator, as well as a method of forming the sheet product. The method comprises forming a mixture of a polyolefin and a fluid having a high vapor pressure, shaping the mixture into a sheet material and subjecting the sheet material to stretching/fluid vaporization at high temperature to form an intermediate material having a ratio of percent fluid to percent polymer crystallinity of between 0.15 and 1, followed by a second stretching/fluid vaporization at a lower temperature while removing a portion of the remainder of the fluid from the sheet. The resultant sheet is annealed and the remainder of fluid is removed to form a sheet product having a thickness comprising a stratified structure of small and larger pore layered configuration across its thickness.

Another known type of separator is disclosed in U.S. Pat. No. 8,722,231 B2, inventors Brilmyer et al., which issued May 13, 2014, and which is incorporated herein by reference. In the aforementioned patent, there is disclosed a separator for a lead-acid energy storage cell. The separator includes a microporous matrix of pore forming particles or fibers, the pore forming particles or fibers being made of natural and synthetic rubbers, polyolefins (such as polyethylene), and non-woven glass fibers. The separator further includes a reversible porosity-controlling agent randomly distributed throughout the microporous matrix. The reversible porosity-controlling agent may be selected from particles that expand or contract in response to an electrolyte concentration or materials that expand or contract in response to temperature. The separator may also include a particulate filler, which may be selected from carbon black, diatomaceous earth and silica particles.

In U.S. Pat. No. 5,955,187, inventors McCormack et al., which issued Sep. 21, 1999, and which is incorporated herein by reference, there is disclosed a self-regulating breathable microporous film layer that transmits water vapor at normal use conditions, and reduces or eliminates the vapor transmission when the vapor in the environment becomes excessive. The film layer includes a voided polymer matrix and a plurality of fine water swellable filler particles disposed within the voids. When there is an excess of vapor, the filler particles swell to block or partially block transmission of vapor through the voids and the film layer. Examples of swellable filler particle materials include superabsorbent, crosslinked hydrogel polymers having an average particle diameter of about 0.1 to 7.0 microns. The self-regulating skin layer may be sandwiched between two skin layers in a multilayer film. The skin layers may include water vapor-permeable polymers, such as ethylene vinyl acetate or ethylene methyl acrylate.

In U.S. Patent Application No. US 2004/0265565 A1, inventors Fischer et al., which published Dec. 30, 2004, and which is incorporated herein by reference, there are disclosed microporous articles that are formed by solid-liquid phase separation from a diluent in combination with a crystallizable thermoplastic polymer, flame retardant and a hindered amine synergist providing novel flame retardant articles. Examples of a crystallizable polymer and a diluent include polyethylene with mineral oil or mineral spirits. Useful flame retardants include halogenated organic compounds, where the flame retardant particles should not inhibit the crystal nucleation of the polymer component during phase separation such that the microstructure grows so large as to adversely weaken the film.

In U.S. Patent Application No. US 2012/0288695 A1, inventor Jenkins, which published Nov. 15, 2012, and which is incorporated herein by reference, there is disclosed a breathable multi-microlayer film material that includes a plurality of alternating coextruded first and second microlayers, wherein the first microlayers comprise an unfilled first polymer composition, and further wherein the second microlayers comprise a second polymer composition and filler particles. The multi-microlayer films have increased breathability but are liquid impermeable, and retain their integrity and strength during processing and use. Particles of superabsorbent polymers may be incorporated in the second microlayer, and the addition of such filler particles may disrupt the local uniformity and orientation of adjacent microlayers while increasing the breathability of the multi-microlayer film and enhancing the ability of the multi-microlayer film to immobilize fluid. The filler particles of the second microlayer may constitute between 10% to 90% by weight of the film and may have a particle size less than 3 microns.

In U.S. Patent Application No. US 2017/0166716 A1, inventor Yen, which published Jun. 15, 2017, and which is incorporated herein by reference, there are disclosed a microporous sheet product and methods of making and using the same. In one embodiment, the microporous sheet product is made by a process that includes melt-extruding a sheet material using an extrusion mixture that includes a thermoplastic polymer, a superabsorbent polymer, and a compatibilizing agent. After extrusion, the compatibilizing agent may be removed from the sheet material. When the sheet product is imbibed with a polar or ion-containing liquid, the superabsorbent polymer swells, causing a reduction in the pore size of the sheet product. The exposure also causes some of the superabsorbent polymer to migrate to the exterior of the microporous sheet product. The microporous sheet product may be used, for example, as a battery separator, as a food packaging material, as a diffusion barrier in the ultrafiltration of colloidal matter, and in disposable garments.

### SUMMARY OF THE INVENTION

It is an object of the present invention is to provide a novel porous sheet product. According to one aspect of the invention, there is provided a porous sheet product, the porous sheet product made by a method comprising (i) providing an extrusion mixture, the extrusion mixture comprising a polymeric binder and a superabsorbent polymer, the superabsorbent polymer being in particle form and having a particle size; (ii) milling the superabsorbent polymer such that the particle size of the superabsorbent polymer, before and after milling, is reduced by a ratio of at least 5:1, (iii) melt-extruding the extrusion mixture to form a sheet material in film form; and (iv) cooling the sheet material.

In a more detailed feature of the invention, the milling step may be performed before the melt-extruding step.

In a more detailed feature of the invention, the milling step may be performed concurrently with the melt-extruding step.

In a more detailed feature of the invention, the milling step may be performed while the superabsorbent polymer is present in the extrusion mixture.

In a more detailed feature of the invention, the milling step may be performed prior to providing the extrusion mixture.

In a more detailed feature of the invention, the polymeric binder may comprise a thermoplastic polymer, the thermoplastic polymer forming an open-celled matrix in the porous sheet product.

In a more detailed feature of the invention, the thermoplastic polymer may comprise at least one member selected from the group consisting of polyolefins, fluoropolymers, polyamides, polyethylene terephthalate, polyacrylics, polyvinyl acetate, polyvinyl alcohol, cellulosics, hydroxypropyl cellulose, polyacrylonitrile, polystyrene, polyurethane, polycarbonate, polysulfone, and polyimide.

In a more detailed feature of the invention, the thermoplastic polymer may comprise at least one member selected from the group consisting of elastomers of polyamide, polyolefin, polyether, polyisobutylene, polybutadiene, polystyrene, atactic polypropylene, polyurethane, ethylene-propylene rubber, ethylene-vinyl acetate copolymer, polyvinyl chloride, oxidized polyethylene, coumarone-indene resins, and terpene resins.

In a more detailed feature of the invention, the thermoplastic polymer may constitute about 1-80% by volume of the extrusion mixture.

In a more detailed feature of the invention, the superabsorbent polymer may comprise at least one member selected from the group consisting of cross-linked polyacrylates, acrylic acid polymers, methacrylates, polyacrylamides, carboxymethyl celluloses, polyvinyl alcohol copolymers, polyethylene oxides, cellulose, starch-grafted copolyacrylates or polyacrylamides, ethylene maleic anhydride copolymers, and copolymers thereof.

In a more detailed feature of the invention, the superabsorbent polymer may have an initial, un-milled, dry size of about 20 microns to 5 mm.

In a more detailed feature of the invention, the superabsorbent polymer, prior to swelling or milling, may be added to the extrusion mixture and may constitute about 0.1-40% by volume of the extrusion mixture.

In a more detailed feature of the invention, the milling step may comprise wet-milling the superabsorbent polymer.

In a more detailed feature of the invention, the method may further comprise, prior to the milling step, swelling the superabsorbent polymer.

In a more detailed feature of the invention, the superabsorbent polymer, after swelling, may be at least twice as large in any of length, width, and height dimensions as the superabsorbent polymer, prior to swelling.

In a more detailed feature of the invention, the swelling step may comprise exposing the superabsorbent polymer to a swelling agent, whereby the superabsorbent polymer may absorb at least a portion of the swelling agent.

In a more detailed feature of the invention, the swelling agent may comprise at least one of water and a C1 to C3 alcohol or diol.

In a more detailed feature of the invention, the method may further comprise, after forming the sheet material, removing at least a portion of the swelling agent from the sheet material.

In a more detailed feature of the invention, the swelling step may further comprise exposing the superabsorbent polymer to an infused agent, whereby the superabsorbent polymer may absorb at least a portion of the infused agent.

In a more detailed feature of the invention, the infused agent may be at least one member selected from the group consisting of humectants, antistatic additives, ionic conductivity additives, color additives, flavorings, medications, and indicators.

In a more detailed feature of the invention, the infused agent may be present in an amount constituting about 0.1 to 60% by volume of the porous sheet product.

In a more detailed feature of the invention, the method may further comprise, after forming the sheet material, removing at least a portion of the swelling agent and/or the infused agent from the sheet material.

In a more detailed feature of the invention, the extrusion mixture may further comprise a compatibilizing agent that promotes uniform mixing of the polymeric binder and the superabsorbent polymer at extrusion temperature.

In a more detailed feature of the invention, the compatibilizing agent may constitute about 1-80% by volume of the extrusion mixture.

In a more detailed feature of the invention, the method may further comprise, after forming the sheet material, removing at least a portion of the compatibilizing agent from the sheet material.

In a more detailed feature of the invention, the extrusion mixture may further comprise an inorganic oxide.

In a more detailed feature of the invention, the porous sheet product may consist of a single layer.

In a more detailed feature of the invention, the porous sheet product may comprise a plurality of layers.

In a more detailed feature of the invention, the porous sheet product may have a thickness of about 0.1-80 mils.

According to another aspect of the invention, there is provided a method of producing a porous sheet product, the method comprising the steps of (i) providing an extrusion mixture, the extrusion mixture comprising a polymeric binder and a superabsorbent polymer, the superabsorbent polymer being in particle form and having a particle size; (ii) milling the superabsorbent polymer such that the particle size of the superabsorbent polymer before and after milling is reduced by a ratio of at least 5:1, (iii) melt-extruding the extrusion mixture to form a sheet material in film form; and (iv) cooling the sheet material.

In a more detailed feature of the invention, the milling step may be performed before the melt-extruding step.

In a more detailed feature of the invention, the milling step may be performed concurrently with the melt-extruding step.

In a more detailed feature of the invention, the milling step may be performed while the superabsorbent polymer is present in the extrusion mixture.

In a more detailed feature of the invention, the milling step may be performed prior to providing the extrusion mixture.

In a more detailed feature of the invention, the milling step may be a wet-milling step, and the method may further comprise swelling the superabsorbent polymer with a liquid swelling agent.

According to still another aspect of the invention, there is provided a porous sheet product, the porous sheet product being made by a method comprising (a) wet-milling an extrusion mixture, the extrusion mixture comprising an elastomer, a superabsorbent polymer, and a swelling agent, at least a portion of the swelling agent being present within the superabsorbent polymer, the swelling agent having a boiling temperature, wherein the wet-milling is performed at a temperature below the boiling temperature of the swelling agent and at a shear rate of at least 20 s⁻¹; (b) melt-extruding the extrusion mixture to form a sheet material in film form; and (c) cooling the sheet material.

According to yet another aspect of the invention, there is provided a porous sheet product, the porous sheet product being made by a method comprising (a) wet-milling a first mixture, the first mixture comprising a superabsorbent polymer, a swelling agent, and an infused agent, wherein the superabsorbent polymer is in particle form and has interstices, wherein the swelling agent and the infused agent form a one-phase homogeneous solution that is absorbed into the interstices of the superabsorbent polymer, wherein the wet-milling is such that the particle size of the superabsorbent polymer, before and after wet-milling, is reduced by a ratio of at least 5:1; (b) melt-extruding a second mixture to form a sheet material in film form, the second mixture comprising the first mixture and a polymeric binder; and (c) cooling the sheet material.

According to a further aspect of the invention, there is provided a porous sheet product, the porous sheet product being made by a method comprising (a) providing a liquid coating mixture, the liquid coating mixture comprising a superabsorbent polymer, a polymer binder, and a swelling agent, the superabsorbent polymer being in particle form and having a particle size, at least a portion of the swelling agent being taken up by at least some of the superabsorbent polymer to swell at least some of the superabsorbent polymer; (b) coating the liquid coating mixture onto a porous substrate; and (c) then, removing at least a portion of the swelling agent from the coated porous substrate, causing the particle size of the swelled superabsorbent polymer to shrink and creating a layer of porosity.

In a more detailed feature of the invention, the liquid coating mixture may be a homogeneous one-phase solution.

In a more detailed feature of the invention, the superabsorbent polymer may be a water-absorbent superabsorbent polymer, the polymer binder may be a water-soluble polymer binder, and the superabsorbent polymer and the polymer binder may be in a water solution.

In a more detailed feature of the invention, a first portion of the swelling agent may be bound swelling agent taken up by the superabsorbent polymer, and a second portion of the swelling agent may be unbound swelling agent not taken up by the superabsorbent polymer.

In a more detailed feature of the invention, the ratio of unbound swelling agent to bound swelling agent may range from 0.1:1 to 10:1.

In a more detailed feature of the invention, the ratio of unbound swelling agent to bound swelling agent may range from 0.2:1 to 3:1.

In a more detailed feature of the invention, the ratio of unbound swelling agent to bound swelling agent may range from 0.3:1 to 1.5:1.

In a more detailed feature of the invention, the liquid coating mixture may have a surface tension below 50 mN/m.

In a more detailed feature of the invention, the liquid coating mixture may have a surface tension below 40 mN/m.

In a more detailed feature of the invention, the liquid coating mixture may have a surface tension below 30 mN/m.

In a more detailed feature of the invention, the liquid coating mixture may have a viscosity in a range of about 50 cps to 50,000 cps.

In a more detailed feature of the invention, the liquid coating mixture may have a viscosity in a range of about 100 cps to 5,000 cps.

In a more detailed feature of the invention, the liquid coating mixture may be an emulsion comprising an oil-based phase and a water-based phase.

In a more detailed feature of the invention, the superabsorbent polymer may be in the water-based phase, and the polymer binder may be in the oil-based phase.

In a more detailed feature of the invention, the method may further comprise milling the swelled superabsorbent polymer, whereby the superabsorbent polymer may have a rounded corner radius.

In a more detailed feature of the invention, the liquid coating mixture may comprise a combination of swelled superabsorbent polymer and non-swelled superabsorbent polymer.

In a more detailed feature of the invention, the liquid coating mixture may comprise an oil phase comprising cyclic olefin and polyisobutylene in mineral spirits and a water phase comprising water-swelled superabsorbent polymer.

In a more detailed feature of the invention, the liquid coating mixture may be a single-phase homogeneous water solution comprising water as the swelling agent and polyvinyl alcohol as the polymer binder.

In a more detailed feature of the invention, the step of providing the liquid coating mixture may comprise swelling the superabsorbent polymer, then wet-milling the swelled superabsorbent polymer, and then combining the polymer binder and the milled and swelled superabsorbent polymer.

In a more detailed feature of the invention, the porous sheet product may have pores having a pore size of at least 0.01 micron, and at least some of the superabsorbent polymer may be embedded within the pores.

In a more detailed feature of the invention, the porous sheet product may have pores, and a size ratio of the pores to the superabsorbent polymer may be at least 2:1.

In a more detailed feature of the invention, the size ratio of the pores to the superabsorbent polymer may be at least 5:1.

The present invention is also directed at a method of separating the electrodes of a battery. In one embodiment, the method may comprise positioning, between the electrodes, a porous sheet product made by a method comprising (a) premixing a superabsorbent polymer with a swelling agent, optionally an infused agent, wherein the superabsorbent polymer is in particle form, wherein the swelling agent is absorbed by the superabsorbent polymer, and wherein the swelling agent enlarges and softens the superabsorbent polymer, (b) reducing the particle size of the superabsorbent polymer by milling, (c) melt-extruding the milled superabsorbent polymer with a thermoplastic polymer and, optionally a compatibilizing agent, to form a sheet material, and (d) forming micropores in the sheet material and optionally removing the swelling agent or infused agent or compatibilizing agent or any combination the agents from the sheet material. In another embodiment, an elastomer may be selected as the polymer used in the melt-extrusion. The extrusion temperature may be carried out below the boiling point temperature of the swelling agent.

The present invention is further directed at a method of packaging a food item. In one embodiment, the method may comprise directly or indirectly contacting the food item with a porous sheet product made by a method comprising melt-extruding a mixture to produce a sheet material, the extrusion mixture comprising a thermoplastic polymer, a superabsorbent polymer containing any combination of swelling agent, infused agent or compatibilizing agent, with the superabsorbent polymer being in particle form, reduced in size by wet milling, and the superabsorbent polymer particles evenly distributed in the final sheet product. An infused agent may be absorbed into the superabsorbent polymer, and optionally desorbed from the sheet product and absorbed onto a packaged item.

Additional objects, as well as aspects, features and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. These drawings are not necessarily drawn to scale, and certain components may have undersized and/or oversized dimensions for purposes of explication. In the drawings wherein like reference numerals represent like parts:

FIG. 1 is a scanning electron microscope (SEM) image, taken from a top view, of conventional superabsorbent particles manufactured as-is, prior to a milling process;

FIG. 2A is a schematic representation of a porous sheet material, showing swelled superabsorbent polymer particles coated into a nonwoven substrate, the swelled superabsorbent polymer particles occupying the volume between the fibers of the nonwoven substrate;

FIG. 2B is a schematic representation of a porous sheet product, where the superabsorbent polymer particles are milled, dried, and reduced in particle size, thereby creating open pore volume within a nonwoven substrate, the available porosity permitting liquid to be imbibed or allowing the transport of gas or liquid;

FIG. 3 is a scanning electron microscope (SEM) image, showing a cross-sectional view of Sample A described in the Examples, where water within the superabsorbent polymer of Sample A is removed, thereby forming open porosity;

FIG. 4 is a mercury porosimetry measurement of the aforementioned Sample A; and

FIG. 5 is a bubble point porosimetry measurement of the aforementioned Sample A.

### DETAILED DESCRIPTION OF THE INVENTION

The present invention is directed at a novel porous sheet product, as well as being directed at methods of making and using the same. As noted above, certain conventional porous sheet products, such as the porous sheet product disclosed in U.S. Patent Application No. US 2017/0166716 A1, include particles of a superabsorbent polymer (SAP) that are dispersed in an open-celled matrix of thermoplastic polymer. The porous sheet product of the present invention may be of a generally similar type; however, the particle size of the superabsorbent polymer in the porous sheet product of the present invention is preferably reduced as compared to the particle size of the superabsorbent polymer in the aforementioned conventional porous sheet product. Such a reduction in the particle size of the superabsorbent polymer may be achieved, for example, by swelling and wet-milling the superabsorbent polymer particles and then melt-extruding an extrusion mixture to form a sheet product, the extrusion mixture including the swelled and wet-milled superabsorbent polymer particles, as well as a thermoplastic polymer and, optionally, a compatibilizing agent. Thereafter, the superabsorbent polymer particles present in the sheet product may be dried, thereby producing a porous sheet product that is permeable to liquid and/or gas. Surprisingly, such a sheet product has properties that are unexpected in view of those of the above-described corresponding conventional sheet product.

In at least one embodiment of the present invention, the swelling agent used to swell the superabsorbent polymer particles may be, but is not limited to, water. In at least one embodiment of the present invention, in addition to using a swelling agent, an infused agent may also be absorbed into the superabsorbent polymer particles. In at least one embodiment of the present invention, the swelling agent may be removed from the swelled superabsorbent polymer particles during sheet material formation whereas the infused agent may remain in situ within the superabsorbent polymer particles as part of the final porous sheet product. Alternatively, the infused agent may also be removed from the swelled superabsorbent polymer particles.

The finished porous sheet product may be a “wet” porous structure or a “dry” porous structure. A “wet” porous structure can be achieved, for example, by having the swelling agent and/or the infused agent substantially occupy and remain in the entire pore volume of the porous sheet material. In certain instances, for example, where the porous sheet product is used as a food packaging material, the porous sheet product may be used with the swelling agent and/or the infused agent remaining in situ, without any further processing after extrusion of the sheet material. In other instances, for example, where the porous sheet product is used as a battery separator, the porous sheet material may be processed after extrusion to remove at least some of the swelling agent and/or the infused agent from the sheet material, thereby producing a “dry” porous structure. In any event, whether the porous sheet product is “wet” or “dry,” the porous sheet product preferably comprises an open-celled matrix of thermoplastic polymer in which superabsorbent polymer particles are dispersed, and the aforementioned porous sheet product is preferably pinhole free.

As noted above, one of the objectives of the present invention is to reduce the size of the superabsorbent polymer particles present in the porous sheet product so as to produce a micro-particle size superabsorbent film, instead of a macro-particle size superabsorbent film. This may be done, according to one embodiment of the present invention, by specially treating the superabsorbent polymer particles, for example, by milling the superabsorbent polymer particles prior to or during extrusion. In at least one embodiment of the invention, the superabsorbent polymer particles may first be allowed to absorb a swelling agent to induce the superabsorbent polymer particles into a so-called “wet” state, wherein the superabsorbent polymer particle hardness may be reduced. Thereafter, the particles may be “wet” milled so that the superabsorbent polymer particle size can be easily reduced.

Furthermore, in at least one embodiment, the thermoplastic polymer that is included in the extrusion mixture may be specifically selected to allow extrusion of the mixture at a temperature that is below the boiling point of the swelling agent. More specifically, the thermoplastic polymer processing temperature may be below the swelling agent boiling temperature by 2° C., more preferably by 6° C., and even more preferably by 12° C. After the sheet material containing the superabsorbent polymer particles has been formed, the swelling agent may optionally be removed, for example, by extraction or drying. Alternatively or additionally, the superabsorbent polymer particles may be post-treated with low pH or a salt solution to reduce the particle size of the superabsorbent polymer particles, thereby creating additional porosity within the formed porous sheet product. Without wishing to be limited to any particular theory behind the invention, the aforementioned low pH or salt post-treatment may serve to replace the alkaline ion in the superabsorbent polymer particles and/or to deactivate the superabsorbent polymer absorbency and/or to encourage the release of absorbed swelling or infused agent. The aforementioned low pH post-treatment may involve a pH below 6 and preferably involves a pH between 1 and 5. For some applications, the swelling agent may be substantially removed, i.e., above 90% removed, preferably above 99% removed, and more preferably above 99.9% removed. Such removal of the swelling agent may be achieved by drying the sheet material containing the swelling agent, for example, using a drying chamber. Where such drying involves using a drying chamber, the partial vapor pressure of the swelling agent within the drying chamber may be below 600 mm Hg, preferably below 400 mm Hg, more preferably below 200 mm Hg, and even more preferably below 100 mm Hg. The temperature of the drying chamber may be above the swelling agent boiling temperature, but preferably is 2° ° C. below the swelling agent boiling temperature, and more preferably is 5° ° C. below the swelling agent boiling temperature.

Without wishing to be limited to any particular theory behind the invention, it is believed that, in general, the smaller the particle size of the superabsorbent polymer, the more evenly the superabsorbent polymer tends to distribute throughout the porous sheet. In accordance with the present invention, there are various ways for achieving a superabsorbent polymer particle of reduced particle size. For example, in one embodiment, the superabsorbent polymer may be wet-milled, dried, and then incorporated into a porous membrane.

Typically, in porous sheet products like that of U.S. Patent Application Publication No. US 2017/0166716 A1, superabsorbent polymer particles on the outer surfaces of the sheet product tend to enlarge after absorbing a polar liquid and, thus, remain on the outer surfaces of the matrix of the sheet product. Moreover, the exposure of the sheet product to a polar liquid also causes some of the superabsorbent polymer to migrate to the outer surfaces of the sheet product. By contrast, due to the fine micron or submicron size of the superabsorbent polymer particles of the porous sheet product of the present invention, the superabsorbent polymer particles of the present invention tend not to migrate from within the matrix of the porous sheet product to the exterior of the matrix. As a result, the porous sheet product of the present invention may tend to swell as a whole. Moreover, the porous sheet product of the present invention may allow water diffusion and may have porosity of at least of 10%, preferably at least 40%, and more preferably above 60%, and may have a pore size below 1000 microns, and preferably below 1 micron for a microporous or nanoporous sheet product. The porous sheet product of the present invention preferably has a tortuous porosity pathway, having tortuosity above 1, preferably above 1.1, more preferably above 1.2, and even more preferably above 1.3.

For purposes of clarity, some of the terms used herein and in the appended claims to describe the subject invention are explained further below:

The term “sheet material” may refer to a unitary article having two large surfaces with respect to its length and breadth dimensions and having a thickness between said surfaces. In general, the term may be used to describe structures achieved during the initial extrusion or shaping of material into a sheet-like form, and of structures produced during subsequent processing of material.

The term “sheet product” may encompass a single-layer or multi-layer structure or membrane consisting of a single sheet material or comprising a plurality of stacks of sheet materials for a designed product use. The product has length and breadth dimensions and has a thickness between said surfaces, and can be porous or non-porous of any pore size, and may be tortuous and free of pinholes.

The term “separator” may refer to a component of a battery, in particular, a storage battery, by which the component maintains a separation between adjacent electrode plates or elements of opposite polarity. The separator may be of various configurations, such as flat (preferred), ribbed, or corrugated sheet, which may be in the form of a membrane or envelope capable of maintaining separation of adjacent electrodes.

The terms “fluid,” “liquid,” or “solvent,” used interchangeably, may refer to liquid components used in the extrusion mixture forming a sheet material. These terms may also be used in reference to a liquid used in a cooling bath for initial cooling of a formed sheet material, fluid used in other processing steps, and for the fluid removed during a stretching and fluid vaporization step.

The term “thermoplastic” may refer to a polymer that becomes pliable, moldable and flows above a specific temperature and returns to a solid state upon cooling. The melt flow rate of a thermoplastic polymer typically increases with temperature. The molecular chains of a thermoplastic polymer typically disassociate on heating and associate through intermolecular forces on cooling and restore the bulk properties. A thermoplastic polymer typically has distinctive melting and glass transition temperatures.

The term “elastomer” may refer to a polymer with viscoelasticity, that is, an ability to resume its original shape when a deforming force is removed. An elastomer typically lacks a distinctive melt temperature and has a very low glass transition temperature; thus, elastomers typically lack drastic phase changes. For purposes of the present application, the term “elastomer” may encompass melt-extrudable or solvent soluble non-cross-linked elastomers, i.e., not thermoset or vulcanized elastomers.

The terms “superabsorbent” and “SAP” may refer to a polymeric compound which can absorb and retain large amounts of liquid relative to its own mass. A superabsorbent polymer typically creates nano-interstices from an absorbing liquid. The as-manufactured particle size of an SAP may be defined as an individual contiguous particle flake or a cluster of particles known as an agglomerate, both types having length, width and thickness dimensions.

The term “swelling agent” may refer to a liquid polar molecule having a hydroxyl group, such as water or a C1 to C3 alcohol or diol having a molecular weight below 100 Da, that can be absorbed into a superabsorbent, uniformly residing within its outer bounds to swell and to reduce the hardness of the superabsorbent at normal temperature and pressure.

The term “infused agent” may refer to a liquid or solid polar molecule containing a combination of at least one functional alcohol, glycol, polyol, hydroxyl, ketone, ether, ester, carbonate, amino, or amide group that forms a single phase solution with the swelling agent and that can be absorbed into a superabsorbent along with at least one swelling agent.

The term “compatibilizing agent” may refer to a liquid at extrusion temperature that promotes compatibilizing and mixing between a thermoplastic polymer and a superabsorbent polymer and that may create micropores in a sheet material by either phase separation or by removal of the compatibilizing agent.

The term “wet-milling” may refer to a processing technique of a superabsorbent polymer particle in which the superabsorbent polymer particle absorbs some amount of a swelling agent and is subsequently exposed to a shear rate and shear force, whereby the superabsorbent polymer particle size may be reduced.

The term “particle size” may refer to a measure of dimension of a solid material having distinct length, width, and thickness dimensions (or X, Y, and Z dimensions, respectively). For purposes of the present application, the largest dimension may be referred to as the length or X dimension, and the shortest dimension may be referred to as the thickness or Z dimension. The specification of a particle size may be the length or largest of any of the three possible dimensions. Additional clarifications of particle size may use the terms smallest, average, mean, median, mode, largest, minimum, maximum, or range.

The term “hardness” may refer to the resistance of a material against local deformation by an indenter, and it may be measured as the reaction force per unit area of some contact area between the indenter and a test material. Typical polymer hardness is measured using a Shore durometer scale. Compressibility and compression force may also be used to represent harness of a superabsorbent polymer in powder form.

The term “pinhole” may refer to a straight through opening in a sheet through the thickness dimension of the sheet, where the opening is without curvature and has a tortuosity of 1. This opening may allow a ray of light to travel and pass through without deflection.

### Materials

The thermoplastic polymer of the above-described extrusion mixture may be used primarily as a binder to provide a supporting scaffold or matrix in which superabsorbent polymer particles may be dispersed and to protect the superabsorbent polymer particles from thermal degradation during melt-extrusion. As such, the thermoplastic polymer may comprise one or more thermoplastic polymers of the type that can be used to form a porous sheet. More specifically, the one or more thermoplastic polymers may include one or more thermoplastic homopolymers, copolymers, terpolymers, or combinations thereof. The one or more thermoplastic polymers of the present invention preferably have a weight average molecular weight of from about 20,000 Da to about 8,000,000 Da. Examples of suitable classes of thermoplastic polymers may include, but are not limited to, polyolefins, fluoropolymers, polyamides, polyethylene terephthalate, polyacrylics, polyvinyl acetate, polyvinyl alcohol, cellulosics, hydroxypropyl cellulose, polyacrylonitrile, polystyrene, polyurethane, polycarbonate, polysulfone, polyimide, and the like. A preferred subclass of thermoplastic polymers may include extrudable elastomers comprising, but not limited to, elastomers of polyamide, polyolefin, polyether, polyisobutylene, polybutadiene, polystyrene, atactic polypropylene, polyurethane, ethylene-propylene rubber, ethylene-vinyl acetate copolymer, polyvinyl chloride, oxidized polyethylene, coumarone-indene resins, and terpene resins. The thermoplastic polymer preferably constitutes about 1-80% by volume of the extrusion mixture. The above polymers and other non-thermoplastics may be used for pore formation coating applications, as long as the polymers are water or organic solvent soluble. Other examples of water soluble polymer may be derivatives of cellulose, methyl cellulose and carboxymethyl cellulose.

The superabsorbent polymer of the above-described extrusion mixture may be used, for example, to absorb a swelling agent and to form a part of the porous sheet product. The superabsorbent polymer of the present invention may comprise one or more types of superabsorbent polymer. Examples of suitable superabsorbent polymers may include, but are not limited to, various cross-linked non-water-soluble polymers, such as cross-linked polyacrylates, acrylic acid polymers, methacrylates, polyacrylamides, carboxymethyl celluloses, polyvinyl alcohol copolymers, polyethylene oxides, cellulose, starch-grafted copolyacrylates or polyacrylamides, ethylene maleic anhydride copolymers, and copolymers thereof. The superabsorbent polymer may further include a functional cation, such as a lithium, sodium, or potassium ion, an alkaline earth metal ion, or a zinc ion.

The aforementioned superabsorbent polymer is preferably in particle form and typically has a definitive length, width, and thickness, or corresponding X, Y, and Z dimensions, both in wet and dry forms. Preferably, the superabsorbent polymer has an initial, un-milled dry particle size, along the largest identified dimension, of about 20 microns to 5 mm. Though wet-milling may be the most efficient method for particle size reduction of the superabsorbent polymer, some applications and products may use un-milled or pre-milled superabsorbent polymer, of any particle size as supplied by a manufacturer.

As described further below, the superabsorbent polymer has a pre-milled dry and wet particle size and a post wet-milled dry and wet particle size. In processing, the wet particle size is larger than the dry non-swelled particle size, preferably by at least 2 times in any of the length, width or thickness dimensions, and more preferably by at least 3 times in any of the length, width or thickness measurements. Organic acid superabsorbent polymers, such as, but not limited to, polyacrylic acid, polymethacrylic acid, and ethylene maleic anhydride polymer, tend to best absorb liquid near a neutral pH. The extent of cross-linking in the superabsorbent polymer should be kept within specific limits so that the superabsorbent polymer may absorb liquids, such as a liquid electrolyte, without forming an amorphous gel. Preferably, the dry superabsorbent polymer constitutes about 0.1-40% by volume of the extrusion mixture. Additionally, the dry superabsorbent polymer preferably constitutes about 0.1-9% by volume in a coating solution, more preferably about 0.1-4% by volume in a coating solution, and even more preferably about 0.1-2% by volume in a coating solution.

The swelling agent of the present invention may be used to reduce the hardness of the superabsorbent polymer particle and also may be used to swell and to enlarge the superabsorbent polymer particle to facilitate its reduction in size via a wet-milling process. The swelling agent may comprise one or more types of swelling agent. A preferred swelling agent may be water that has been de-ionized, with certain halogen and metal ions substantially removed, such as chlorine, bromine, calcium, iron, tin, and antimony ions. The foregoing halogen and metal ions may be present in the porous sheet product in an amount below 1000 ppm, preferably below 100 ppm, and more preferably below 50 ppm. Certain alcohols may also be used as a swelling agent. The swelling agent may be chemisorbed into the superabsorbent polymer particle, creating nanoporosity within the superabsorbent polymer particle in the process. The swelling agent may be present in the superabsorbent polymer particle in an amount that is at least 200% by weight as compared to the dry superabsorbent polymer particle, preferably at least 600% by weight as compared to the dry superabsorbent polymer particle, more preferably at least 1,800% by weight as compared to the dry superabsorbent polymer particle, even more preferably at least 3,500% by weight as compared to the dry superabsorbent polymer particle, and still even more preferably at least 11,000% by weight as compared to the dry superabsorbent polymer particle. A preferred embodiment of a coating liquid solution may be used to wet-mill the superabsorbent polymer particle below a certain percentage of the swelling agent, yet form a coating solution above this percentage, wherein such a percentage may be 200%, preferably 600%, more preferably 1,800%, and even more preferably 3,500%.

Based on the cubic geometry of the superabsorbent polymer particle, each dimension of the superabsorbent polymer particle cube may double, i.e., increase in size by 100%, if the superabsorbent polymer particle absorbs 800% by weight of the swelling agent. Analogously, the superabsorbent polymer particle may triple in size if it absorbs about 27 times by weight of the swelling agent. A wet particle can be easily reduced in size by mechanical means. The hardness of the superabsorbent polymer particle may be reduced by changing from a dry state to a wet state, i.e., after incorporating the swelling agent. The hardness can be represented by measuring the compression force needed to compress the superabsorbent polymer particle. Once the swelling agent is absorbed into the superabsorbent polymer particle, the force required to compress the swelled superabsorbent polymer particle from 100% volume to 90% volume may be reduced by at least 10%, more preferably by at least 20%, and even more preferably by at least 50%. The swelling agent and the superabsorbent polymer particle may collectively constitute about 10% to 99%, preferably 60% to 95%, by volume of the extrusion or wet-mill mixture.

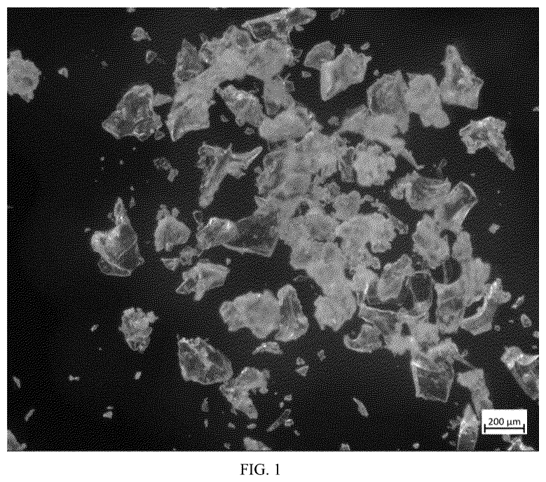

Generally speaking, when a superabsorbent polymer is manufactured, the superabsorbent polymer is typically crystalline with a polytope geometry having at least two flat surfaces or sides with at least one corner angle of less than 90° and contains about 8% water. Due to the manufacturer's crush grinding process, the superabsorbent polymer polytope tends to have flat sides and sharp corners, as shown in FIG. 1. The superabsorbent polymer of FIG. 1 may be dry-crushed, i.e., processed through a pair of nip rollers set to a 50 micron gap. The crushed superabsorbent polymer tends to have a flake-like appearance and may then be sifted through filters to produce particles having a particle size below 1 mm. The foregoing type of mechanical dry-crushing can be energy intensive and typically results in equipment wear. It is not possible to use dry-crushing to produce superabsorbent polymer particles of submicron particle size for thin film formation for at least the reason that a small nip gap of under 50 microns is exceedingly difficult to control. By contrast, wet-milling of superabsorbent polymer particles is easy and energy efficient, as the durometer of the superabsorbent polymer particle is significantly reduced when wet. For a wet-milling process, the superabsorbent polymer and the swelling agent can collectively constitute 100% of the wet-milled composition, i.e., no polymer binder present. On the other hand, with a polymer binder included, the superabsorbent polymer and the swelling agent can collectively constitute up to 99% of the wet-milled composition.

In another embodiment of the invention, the porous sheet product may comprise, in a single layer, a combination of a swelled superabsorbent polymer and a non-swelled superabsorbent polymer. Such a combination of swelled and non-swelled superabsorbent polymers may serve to introduce a matrix of open cell porosity and/or to maximize absorption capability of the membrane. Alternatively, the porous sheet product may be constructed to include a layer comprising a swelled superabsorbent polymer and an adjacent layer comprising a non-swelled superabsorbent polymer. Both of the foregoing approaches may achieve a bimodal or multimodal particle size distribution, having modes of larger and smaller superabsorbent polymer particle sizes and resultant pore sizes. This result can be accomplished, for example, by blending un-milled and wet-milled superabsorbent polymer particles and preferably having fewer un-milled particles than wet-milled particles.

A preferred mixture used to form a porous sheet material may consist of or comprise a single-phase liquid slurry solution, preferably water-absorbed superabsorbent polymer in a water solution, preferably free of compatibilizing agent, more preferably with a water-soluble polymer binder. In this case, a maximum amount of water may be absorbed into the superabsorbent polymer, with additional water and water-soluble polymer forming a homogeneous one-phase solution. The aforementioned solution may then be coated onto a substrate to form a porous sheet product. A viscosity modifier may be used to achieve a desired viscosity for coating. Another embodiment comprises two liquid phases, such as a water-in-oil or oil-in-water emulsion, where the superabsorbent polymer is in the water phase, thereby resulting in a superabsorbent polymer in a polymer porous membrane. An advantage of having a continuous oil polymer phase is to form smaller surface pores, while encapsulating internal water phasing, with superabsorbent polymer creating larger internal pores for higher porosity, enabling higher electrolyte permeation. This dual pore size structure produces a mode of large pores and a mode of small pores, with the resulting porosimetry exhibiting a bi-modal pore size distribution. Depending on the application of the sheet product, the ratio of larger to smaller pore sizes may be from 2:1 to 2,000:1, preferably from 5:1 to 500:1, and more preferably from 10:1 to 100:1.

As noted above, an infused agent may also be absorbed into and retained within the nanoporosity of the superabsorbent polymer during wet-milling and may be used to add functionality to the porous sheet product. The infused agent may comprise one or more types of infused agent. Examples of suitable infused agents may include humectants, such as glycerin; antistatic additives; ionic conductivity additives, such as alkali salts; color additives, such as caramel; flavorings; medications; indicators; or other additives. The infused agent may be present in the final porous sheet product in an amount constituting about 0.1% to 60% of the final porous sheet product and may be present in an amount constituting at least 5%, preferably at least 25%, more preferably at least 45%, and even more preferably at least 100%, as compared to the superabsorbent polymer. The weight ratio of the infused agent to the swelling agent in the porous sheet material may be below 2:1 and preferably ranges from 0.1:1 to 1:1, more preferably from 0.1:1 to 0.5:1.

The compatibilizing agent of the present invention may be, for example, any plasticizer or surfactant that promotes uniform mixing of the thermoplastic polymer and the superabsorbent polymer at extrusion temperature. The compatibilizing agent may comprise one or more compatibilizing agents. The compatibilizing agent may be a liquid or solid at room temperature. Examples of materials that may be used as a compatibilizing agent according to the present invention may include, but are not limited to, low molecular weight organic liquids, such as mineral spirits, mineral oil, lower molecular weight alkanes, C6 to C20 aliphatic, alicyclic or aromatic hydrocarbons, polyethylene oxide, glycols (e.g., polypropylene glycol), hydroxypropylene, phthalates, oils, surfactants or the like, as well as mixtures thereof.

In those instances where, for example, the porous sheet material is to be used as a food packaging material, no further processing of the porous sheet material may be needed after extrusion and film formation. On the other hand, in those instances where, for example, the porous sheet material is to be used as a battery separator, the porous sheet material may thereafter be treated so that at least some of the compatibilizing agent is removed from the porous sheet material. Such removal of the compatibilizing agent may be effected, for example, by a conventional solvent extraction technique and/or by the stretching/vaporization technique of U.S. Patent Application Publication No. US 2013/0029126 A1. The removal of the compatibilizing agent in the aforementioned fashion may create open capillaries, available for electrolyte absorption and conductivity of a sheet product. The compatibilizing agent preferably constitutes about 1-80% by volume, more preferably 10-50% by volume, of the extrusion mixture.

The above-described extrusion mixture may further comprise one or more fillers, anti-oxidants, stabilizers, and the like. For example, the extrusion mixture may contain one or more inorganic oxides, which may improve the porosity and rate of swelling of the sheet product. Suitable inorganic oxides may include, but are not limited to, oxides of silicon, aluminum, lithium, magnesium, calcium, titanium, zinc, zirconium, or barium. Such oxides may be in the form of fine particles, preferably less than 50 microns in diameter, likely from 0.01 microns to 20 microns. Such particles may have a surface area of at least 5 m²/g, and may have a pore volume (BET) of about 0.01-1 ml/g. The particles may be prepared by any method that results in the production of fine particles, such as, but not limited to, milling, condensation, precipitation, fumed, or any other appropriate method. The foregoing inorganic oxides, when added to the extrusion mixture, tend to increase porosity, especially after stretching of the polymer sheet material. Where an inorganic oxide is used as part of the extrusion mixture, the inorganic oxide preferably constitutes about 1-70% by volume of the mixture. Typical inorganic oxides have a specific gravity of 2 to 10; thus, an amount constituting 70% by volume may be equivalent to 94% by weight.

An alternative method for superabsorbent polymer particle size reduction may be the use of ball milling technology, wherein a filler can act as the milling ball. In any case, whether dry milling or wet milling is employed, inorganic oxides may be used for milling of the superabsorbent polymer. A preferred size ratio of the inorganic oxide to that of the milled and dried superabsorbent polymer may be from 1:1 to 500:1, more preferably from 1:1 to 100:1, even more preferably from 1:1 to 50:1.

### Processing Method

As noted above, one aspect of the present invention may involve the use of an extrusion milling process in the production of a porous sheet material. Depending on the intended use of the porous sheet material, the porous sheet material may thereafter undergo additional processing. Further details are provided below.

In one embodiment, a dry-blend master-batch of the extrusion mixture may be prepared, wherein the extrusion mixture may include superabsorbent polymer particles that are comparatively hard. A swelling agent, such as water, may then be added to the extrusion mixture, whereby the superabsorbent particles in the extrusion mixture may be exposed to and may absorb the swelling agent. Due to their absorption of the swelling agent, the superabsorbent polymer particles may undergo a reduction in hardness. Subsequently, the foregoing mixture, including the swelled superabsorbent polymer particles, may be wet-milled. Such wet-milling may be effected using a mechanical shearing and milling device, for example, a homogenizer, a static mixer, a Banbury mixer, or an extruder. The aforementioned milling of the superabsorbent polymer particles and the extrusion of the extrusion mixture may be performed in a single operation or may be performed in a series of separate processing steps. The various components of the extrusion mixture may be fed into a single or twin-screw feed chamber of an extruder. An example of a suitable extruder is disclosed in PCT International Publication No. WO 2009/051278 A2, which was published on Apr. 23, 2009, and which is incorporated herein by reference.

Alternatively, the dry superabsorbent polymer and the thermoplastic polymer may be fed into the first feed barrel of an extruder as a first feed stream, and the swelling agent or a combination of swelling and infused agents may be injected into a downstream barrel from the feed barrel as a second feed stream to swell and to soften the superabsorbent polymer within the extruder for wet-milling.

The first heated barrel of the extruder may be set below the boiling temperature of the swelling agent. Preferably, the first heated barrel of the extruder is set 2° C. below the boiling temperature of the swelling agent, more preferably 6° ° C. below the boiling temperature of the swelling agent, even more preferably 12° C. below the boiling temperature of the swelling agent, and still even more preferably 20° ° C. below the boiling temperature of the swelling agent. Where water is used as the swelling agent, the first heated barrel may be set at a temperature that is below 100° ° C., more preferably a temperature that is below 98° C., even more preferably a temperature that is below 88° C., and still even more preferably a temperature that is below 80° C. Optionally, at least one melt seal can be created within an extruder zone, where one or more barrels may be heated above the boiling point of the swelling agent and the swelling agent may be superheated, creating a higher than atmosphere pressure zone. The pressure within this higher than atmosphere pressure zone may be above 1.1 bar absolute, more preferably above 1.5 bar absolute, even more preferably above 3 bar absolute.

A liquid compatibilizing agent may be injected into the aforementioned barrel zone to improve the melting of the thermoplastic polymer. Subsequently and optionally, an open barrel may be used as an evacuation zone to decompress, evacuate, or vacuum the mixture at or below atmospheric pressure to remove potential volatiles, such as water vapor in the mixture. The pressure within this evacuation zone may be below 1.0 bar absolute, more preferably below 0.9 bar absolute, even more preferably below 0.7 bar absolute, and still more preferably below 0.5 bar absolute. The aforementioned vacuuming step may serve to remove a majority of the swelling agent volatiles, resulting in a wet-milled superabsorbent polymer having less than 10%, preferably less than 5%, and more preferably less than 2%, of the swelling agent in the extrudate mixture. The melted material may then be pumped through a heated die, which may be used to form the extrudate and sheet material. Depending on the end product, the pumping barrels and die may be set at below the boiling temperature of the swelling agent, similar to the feed barrel temperature condition previously described. Where water is used as the swelling agent, the extrusion die may be set at a temperature that is below 100° C., preferably below 98° C., more preferably below 94° C., even more preferably below 88° ° C., and still even more preferably below 80° ° C. In order to increase the shear efficiency in milling, the extruder or other milling device preferably applies at least 2 bars of pressure, more preferably 11 bars of pressure, even more preferably 50 bars of pressure, and still even more preferably at least 110 bars of pressure. Process pressure can be equated to milling shear stress since both are in units of Pascal. Higher pressure ensures the intimate contact of superabsorbent polymer particles for shearing by extrusion, ball milling or homogenizing.

In order to reduce the size of the superabsorbent polymer particle via wet-milling, the extruder preferably has a collection of kneading blocks with a total kneader block length (L) to screw diameter (D) ratio of at least 0.5 L/D, more preferably at least 1.4, and even more preferably at least 2.8 L/D, with the total combined kneading block length being above 10%, preferably above 15%, and more preferably above 20%, of the total extruder screw length. As a matter of convention, the extruder has right-handed and left-handed elements, where right is the conveying element and left is the reverse element. To improve the superabsorbent milling effectiveness, the extruder cavity is preferably filled with the superabsorbent polymer mixture, creating a higher pressurized zone and exposing this mixture to a shear rate and corresponding shear force. A preferred extruder screw design has at least one adjoining right and left element pairing to build pressure, more preferably has repeated and multiple right-and-left element pairings (e.g., two such pairings), and even more preferably has three such pairings, for a total combined L/D of at least 0.6, more preferably at least 1.2, and even more preferably at least 1.8. A multiple right-left combination shear zones acts like a filtering device, ensuring a higher and higher percentage of swelled superabsorbent polymer is milled through each additional shearing step.

The mixture of thermoplastic and superabsorbent polymers may form a non-Newtonian fluid. In general, viscosity is a factor that correlates shear rate in s⁻¹ and shear stress in pascal. A preferred viscosity for shearing a sheet material may be at least 100 centipoise or at least 0.1 pascal-second (pas), preferably at least 1 pas, more preferably at least 10 pas, and even more preferably at least 100 pas. A higher shear rate will induce a corresponding higher shear force in milling, i.e., doubling the mixture viscosity will double the stress at the same shear rate. The shear rate in an extruder is affected by the screw design, screw speed in rpm, and equipment clearance of the melt passage. To improve the effectiveness of milling, a larger diameter extruder and a higher extruder screw rpm will increase shear rate, γ, enabling a reduction in the particle size of the superabsorbent polymer. In general, the extruder should operate at above 50 rpm, generating a shear rate of at least 20 s⁻¹ in the extruder, preferably a shear rate of at least 40 s⁻¹, more preferably a shear rate of at least 90 s⁻¹, and even more preferably a shear rate of at least 150 s⁻¹. An equation that describes this extrusion shear rate, γ, is:

γ=2πNR/60h
wherein
N=rotational speed in rpm
R=extruder screw radius, in mm
h=channel depth, or gap where superabsorbent passes through, in mm

The general expression for shear rate between parallel plates is:

γ=v/h
wherein
γ is the shear rate, measured in reciprocal seconds
v is the velocity of the moving plate, in meters per second
h is the distance or gap between the two parallel plates, in meters

In an extruder, shear rate can be increased by reducing the processing gap within the extruder. This can be accomplished by selecting and arranging specific screw design elements, such as having a gap, h, that is less than 0.1 times, preferably less than 0.07 times, more preferably less than 0.05 times, and even more preferably less than 0.03 times, of the screw radius, R. This screw design may also be accomplished by arranging sequential pairing of right-to-left extruder screw elements of conveying, kneading, mixing or shearing elements, where the left and right elements are mirror images of each other, thus creating a reduced gap, h, that significantly increases shear rate, thereby reducing the superabsorbent polymer particle size. Another way to ensure higher shear rate and, thus, reduce superabsorbent polymer particle size is to add a screen filter within the melt passage of the extruder assembly. Such a screen may have a mesh size above 100, preferably above 200, more preferably above 325, and even more preferably above 700. (The mesh size of a screen is defined as the number of wires per one inch of length or width. A 200 mesh filter has a potential theoretical gap (h) of 127 microns or less. In practical terms, such a gap is likely 65 microns or less, thereby producing a shear rate of 248 s″ when superabsorbent polymer material flows at a rate of one meter per minute.)

In any shearing or extrusion process, the swelled superabsorbent polymer may be wet-milled, resulting in a wet average particle size below 100 microns, more preferably below 40 microns, even more preferably below 10 microns, and still even more preferably below 1 micron. A wet-milled superabsorbent polymer particle tends to have rounded corners from the tumbling action of shear milling. This rounded corner may be defined as having a corner radius, which may be at least 0.5% of the longest dimension of the particle, perhaps at least 2% of the longest dimension of the particle, or even 10% of the longest dimension of the particle. The advantage of having rounded corners of superabsorbent polymer particles is to improve the uniform application of coating solution onto a substrate, thus avoiding potential pinholes and protrusions from sharp superabsorbent particle corners in thin sheet product production. Although there is no limitation on the smallest particle size of the superabsorbent polymer particle, the average re-dried particle size of the superabsorbent polymer particle is below 30 microns, more preferably below 3 microns, even more preferably below 0.6 micron, and still even more preferably below 0.3 micron. The largest particle size of the re-dried superabsorbent polymer particle population is to be below 100 microns, preferably below 10 microns, more preferably below 3 microns, even more preferably below 0.9 micron. When comparing the pre-milled dry superabsorbent polymer particles versus the post wet-milled and re-dried SAP particle size, the median and largest particle size may be reduced by a factor of at least 2, preferably by a factor of at least 5, more preferably by a factor of at least 15, and even more preferably by a factor of at least 30. For example, the largest dry superabsorbent polymer particle found before and after wet-milling may be reduced from 200 microns to 13 microns, or even smaller, to 6 microns.

The melted extrudate may be formed by a cast film die or a blown film die. The formed sheet material may then be cooled, for example, by casting the sheet material onto a chilled roll or by immersing the sheet material in a cooling bath to solidify the sheet material. The cooling roll or bath is preferably maintained at a temperature below 100° C. so that the sheet material may be cooled below the melt temperature of the thermoplastic polymer. The film may be used without further processing or, optionally, may be stretched in the machine and/or transverse direction.

In certain instances, for example, where the sheet material is to be used as a food packaging material, it may be acceptable for the infused agent and/or the swelling agent to be retained in situ within the superabsorbent polymer and/or for the compatibilizing agent to remain within the sheet material. In other instances, for example, where the sheet material is to be used as a battery separator, the cooled sheet material may be subjected to some form of processing, which may involve one or more of extraction, vaporization, and stretching, to remove some or substantially all of the swelling agent and/or the infused agent and/or the compatibilizing agent. For example, such processing may involve a fluid vaporization and a stretching of the sheet material in at least one direction. This first direction of stretching may be conducted, for example, in the machine direction from which the sheet material exits the extrusion die head and the cooling bath or rolls. Subsequent stretching may be in a second direction, which may be transverse to the first stretching direction. The combined stretches may have an overall ratio of about 2-70 fold of the original biaxial area. The stretching may be performed in sequential monoaxial steps or in simultaneous biaxial stretches. The sheet material may be optionally annealed and relaxed in one or both stretched directions to improve dimensional stability.

In an alternative embodiment, the shaped sheet material may also be extruded in an annular die, forming the sheet into a continuous tubular form. The stretching orientation may be conducted in a conventional single, double or triple bubble blown film equipment. The tubular film may be longitudinally stretched and simultaneously inflated to orient the film under a specific temperature.

The thickness of the porous sheet product of the present invention may be about 0.1-80 mils (about 0.0025-2.0 mm). A single layer “dry” sheet product according to the present invention may be macroporous, i.e., above 10 microns in pore size, or microporous, i.e., below 1 micron in pore size. The porous sheet of the present invention is capable of absorbing at least 30% by weight of electrolyte or other liquid. The resistivity of the sheet product of the present invention is preferably below 1,000 ohm-cm, more preferably below 100 ohm-cm, in a 30% KOH electrolyte.

The porous sheet product of the present invention may consist of a single layer of the type described above or may comprise a plurality of formed, stacked or laminated layers. A laminate structure may be readily formed using conventional multi-sheet extrusion head devices, e.g., co-extrusion. Examples of multilayer structures are described in European Patent Application Publication No. EP 1 911 352 A1, published Apr. 16, 2008, which is incorporated herein by reference. One or more of the layers of a multilayer structure may be a protective layer, which may be non-porous to limit the permeability of pathogens or other detrimental microorganisms and to improve film durability. Alternatively, the multilayer structure may comprise a middle layer that includes a superabsorbent polymer and porous outer layers that do not include such a superabsorbent polymer.

As another example of a sheet product comprising a superabsorbent polymer of small particle size, a multilayered sheet product according to the present invention may comprise an outer layer that comprises a superabsorbent polymer that has been swelled and milled and also may comprise an internal layer that comprises a superabsorbent polymer that is not swelled and/or that is not milled. The aforementioned outer layer may have dry superabsorbent polymer particles that are below 30 microns in size, preferably below 10 microns in size, more preferably below 5 microns in size, whereas the internal layer may have a superabsorbent particle size that is above 30 microns in size, preferably above 100 microns in size. The ratio of internal layer to outer layer superabsorbent polymer particle size is least 2:1, preferably 5:1, and more preferably 10:1. This multilayered arrangement of superabsorbent polymer particle size creates a microporous outer layer and a highly swellable internal layer that is better able to mitigate transmission of pathogens and to limit superabsorbent polymer particle migration.

As noted above, one aspect of the present invention is to introduce a swelling agent into a superabsorbent polymer particle to soften the superabsorbent polymer particle, thereby allowing a reduction in size of the superabsorbent polymer particle via wet milling and enabling a shaped composite article of superabsorbent polymer to be formed. The swelling agent may also function as a carrier to help absorb and infuse a functional chemical into the superabsorbent polymer particle, such as an antimicrobial agent, a colorant, or a flavor. The formed article may comprise a liquid-filled wet porosity, where the swelling agent may be allowed to remain in-situ after the formation of an article, or the swelling agent (and/or compatibilizing agent if present) may be partially removed to form an open-cell dry porosity. A superabsorbent polymer may also be induced into creating a so-called wet-to-dry porous article. As swelling agent is removed from a superabsorbent polymer in the sheet material, the superabsorbent polymer particle will undergo a reduction in particle size, creating open pore volume and allowing gas or liquid to permeate through the created pores.

In addition to being used as a battery separator, the above-described porous sheet product may be put to other uses. For example, the porous sheet product may be imbibed with a material that endows the porous sheet product with a specific function. The imbibing material may be a liquid or a dispersion of a solid and may include one or more types of imbibing materials. Examples of such imbibing materials may include medicaments, fragrances, flavorings, colorants, antistatic agents, surfactants, antimicrobials, pesticides and solid particulate materials, such as activated carbon and pigments.

Another type of porous sheet product according to the present invention may comprise an open-celled or closed-celled foam. Such a product may be formed by treating, using heat or vacuum, a sheet material comprising a superabsorbent polymer absorbed with a swelling agent and/or an infused agent in such a way as to vaporize the swelling agent and/or the infused agent. The liquid to gas expansion from said vaporization creates a porous foamed network utilizing a moisture popcorn mechanism. In the case of such a foam product, macropores may be produced. Such macropores may be above 50 microns in size, 200 microns in size, or even 1 mm to 10 mm in size.

The porous sheet product of the present invention may be bonded to a variety of substrates to produce a multitude of multilayered structures. Such substrates may be nonwoven, porous, or non-porous sheet materials. Examples of materials suitable for making such substrates may include, but are not limited to, fiber-glass, cellulose, polyolefins, polyamide, polyester and other polymers. Lamination of the porous sheet product to the substrate may be accomplished by conventional techniques, such as coating, impregnation, adhesive bonding, spot-welding, or by other techniques which do not destroy or otherwise interfere with porosity and/or which do not create undesirable pinholes or perforations. Alternatively, a nonwoven substrate, itself, may include fibers made with wet-milled superabsorbent polymer, where the diameter of such fibers to the particle size of the dry superabsorbent particle may have a ratio of at least 2:1, more preferably 5:1, and even more preferably at least 10:1. The nonwoven substrate may be formed by spinning, dry-laid, melt-blown, or spunbond processes. The substrate may be treated to impart hydrophilic or hydrophobic affinity thereto prior to lamination with or coating of sheet material using methods such as corona treatment.

The porous sheet product of the present invention may be employed in any of a wide variety of situations where porous structures may be utilized. For example, the porous sheet product of the present invention may be used in the filtration or desalination of matter, for example, as a reverse osmosis or diffusion barrier. Alternatively, the porous sheet product of the present invention may be used as a geo-membrane, a breathable non-woven protective scrim, a disposable garment, a diaper, gloves, a wound dressing or an artificial skin which takes advantage of moisture absorption and transport from perspiration yet provides a barrier to pathogens and liquid chemicals.

Another application of the porous sheet product of the present invention may be in the field of food packaging. For example, the porous sheet product of the present invention may be used as a packaging for cooked or uncooked cheese, meat, and sausage, to provide moisture and/or flavor transfer to foods and/or to promote adhesion. Alternatively, the porous sheet product of the present invention may be used as a chicken shrink bag or as a fish, beef or pork display tray liner to absorb excess processing fluids. Other applications may include packaging for fresh produce and bread, where the porous sheet product may be used to help achieve equilibrium of moisture, oxygen, and carbon dioxide levels in order to prolong product shelf life.

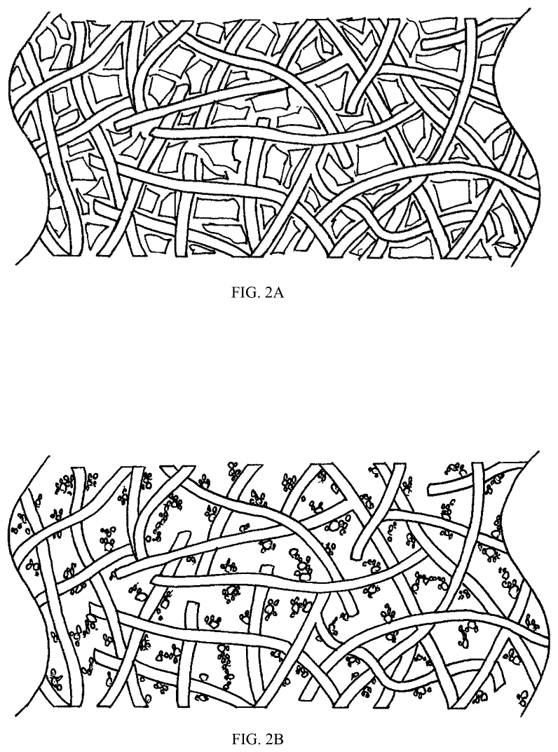

In another embodiment, milled or un-milled superabsorbent polymer particles may be coated onto a lithium battery separator or electrode, i.e., a so-called ceramic coating in a lithium battery assembly. The foregoing ceramic coating layer is intended to add dimensional stability to a lithium battery separator. Since a superabsorbent polymer may absorb up to 500 times of its own weight of water, a swelling agent absorbed superabsorbent polymer can be bound together by a polymer binder, forming a non-permeable structure; however, once this swelling agent is removed by extraction or drying, the superabsorbent polymer particle size will reduce in size, creating open pores and gaseous volume within the sheet product, thereby allowing gas or liquid to diffuse or transport. This phenomenon is shown schematically in FIGS. 2A and 2B. FIG. 2A depicts un-milled superabsorbent polymer particles embedded within a nonwoven substrate whereas FIG. 2B showed wet-milled, dried, and shrunken superabsorbent polymer particles embedded within the nonwoven substrate. As can be seen, the shrunken superabsorbent polymer particles of FIG. 2B create an open porosity after the removal of the swelling agent. Depending on the end use of the product, certain grades of superabsorbent polymer particles do not require milling, regardless of particle size. A superabsorbent polymer particle can be mixed directly into a coating solution, coated onto a substrate and dried. The thus-formed sheet product takes advantage of porosity creation from the shrinking superabsorbent polymer particles. An air filter is such an example, where pores above 50 microns are desirable.

When forming a single-phase liquid coating solution, the solution may comprise bound and unbound liquid. (Swelling agent within a superabsorbent polymer particle may be considered “bound” whereas liquid outside of the superabsorbent polymer particle that provides fluidity for the solution may be considered “unbound.”) The ratio of unbound liquid to bound swelling agent within the superabsorbent polymer may be in the range of 0.1:1 to 10:1, preferably 0.2:1 to 3:1, and more preferably 0.3:1 to 1.5:1. The foregoing coating solution may be in a slurry form where the superabsorbent polymer and any added inorganic materials are in particle form. The coating solution may further comprise a water-soluble organic liquid to modify the surface tension of the coating solution. The surface tension of the coating solution is preferably below 50 mN/m, more preferably below 40 mN/m, and even more preferably below 30 mN/m. One or more of an infused agent, an alcohol, and a surfactant may be incorporated into the coating solution to lower its surface tension. For a water-based coating solution, a preferred polymer binder solubility parameter may be above 11 (cal/cm³)^{1/2}, and a preferred coating solution viscosity may be in the range of about 50 cps to 50,000 cps, and more preferably 100 cps to 5,000 cps.

When a superabsorbent polymer solution is coated onto a porous substrate, such as polyolefin separator, the coating can be made using a coating die, with slot die and thickness being controlled by a doctor blade. A shear force may be applied to ensure the uniformity of the coating. More importantly, a shear force may ensure the lay-flat of a superabsorbent polymer particle. As previously mentioned, a superabsorbent polymer particle has X, Y and Z dimensions, with the Z dimension typically being the smallest of the three dimensions. When compared to the X dimension, the Z dimension may have a ratio of 1:1 or less, more preferably 0.8:1 of less, and even more preferably below 0.5:1. The final sheet product preferably has the plane of the X and Y dimensions oriented parallel to the coated sheet (i.e., so-called lay-flat to the coated separator), where the Z direction is at about a 90° angle from the coated substrate plane. This surprising coating result ensures the minimization of coating thickness. For example, even when a superabsorbent polymer particle is 20 microns in length along the X dimension, as long as the particle's Z dimension is 5 microns or less, a coating that is 5 microns in thickness can be achieved. For a ceramic coating solution, the mean Z dimension of a dry superabsorbent polymer particle may be in the range of 0.001 micron to 2 microns, and preferably in the range of 0.01 micron to 0.5 micron. In order to produce this lay-flat of the superabsorbent polymer particles, a preferred shear rate may be at least 20 s⁻¹, more preferably at least 50 s⁻¹, even more preferably at least 120 s⁻¹, and still even more preferably at least 200 s⁻¹. When a swelling agent incorporated superabsorbent polymer is coated onto a superabsorbent-free lithium battery separator, the dried coated layer may have a median pore size of at least 0.01 micron, preferably at least 0.1 micron, and more preferably at least 1 micron, whereas an internal, uncoated polyolefin membrane may have a median pore size less than 1 micron, and preferably has a median pores size of 0.1 micron or less. The ratio of the median pore size of the surface layer to the internal layer may be at least 2:1, preferably at least 5:1, and more preferably above 10:1. The present invention of a sheet product formed from casting or coating of a liquid solution exhibits a network of interconnected open cell porosity, produced without processing orientation or stretch. The pore size may be least 0.01 micron, while having smaller size superabsorbent polymer particles embedded within the pores. The wet, then re-dried, superabsorbent particles are adhered to the internal pore surface by a polymer, wherein the size ratio of the pore to superabsorbent particle is at least 2, preferably at least 3, more preferably at least 4, and most preferably at least 5. This sheet product has dual pore sizes, with the larger open cell pores enabling fast wicking and transport of liquid, while the smaller superabsorbent particles allow chemisorption of liquid for retention. The resultant sheet product preferably has a Gurley air permeability below 200 second/mil, more preferably below 100 second/mil, of sheet thickness.

Optionally, the above-described coating solution may be coated onto a battery electrode, where this coating may perform as a battery separator. For example, a coating assembly may comprise an anode-coating-separator stack, an anode-coating-separator-coating-cathode stack, or an anode-coating-cathode stack, where the coating may be multilayered and may have a different pore size and porosity in each of the layers. The coating may be designed to provide adhesion of electrodes or stacks, such as a stack-coating-stack, thereby providing intimate electrode separator contact. By removing the swelling agent, a coating of a superabsorbent polymer layer may become porous without being stretched. A superabsorbent polymer may be acidified to replace its cation with a hydrogen ion, in effect making the superabsorbent polymer less hydrophilic for lithium battery applications.

### Examples

The following examples are given for illustrative purposes only and are not meant to be a limitation on the invention described herein or on the claims appended hereto. All parts and percentages given in the description, examples and claims appended hereto are by volume unless otherwise stipulated. Further, all ranges of numbers provided herein above shall be deemed to specifically disclose all subset ranges of numbers within each given range.

In the case of an electrochemical cell separator, an object is to achieve the highest conductivity in electrolyte while demonstrating stable physical and mechanical characteristics. All samples below were processed similarly, with the material mixture processed in the prior described sequence via a co-rotating twin screw extruder. The extruder was set at a temperature of 80° ° C. at the first heated barrel zone and at a temperature of 140° C. at the superheated melt zone. The extruder was vented prior to the pumping section, and the extruder pumping section and die were set at 90° ° C. The melt extrudate was cast onto a cast roller set at a temperature of 40° C., with the total extrusion rate being 4 kg/hr and with the cast roller having a takeoff speed of 4 ft/min. The extruder was 30 mm in diameter, with a screw channel depth of 5 mm and with a pair of right-handed and left-handed elements to decrease gap and to increase shear rate. Additionally, the extruder had a melt clearance gap of 1 mm, and the mixture was extruded at 100 rpm, thus producing a shear rate of 156 s⁻¹. The extruder had a right-to-left L/D ratio of 0.9 and generated a pressure of 3 to 6 bars.

### Materials:

Materials used in the formation of the sheet product include:
Polyethylene—PE-EVA NA362006 from Lyondell Bassell, La Porta, Texas or Vista from Exxon-Mobil
Polyamide—Nylon GRILAMID® from EMS Chemie AG, Domat/EMS, Switzerland Polyisobutylene from BASF SE, Ludwigshafen, Germany Cyclic Olefin from Topas Advanced Polymer Inc., Florence, Kentucky Polyvinyl Alcohol from Clariant GmbH, Sulzbach, Germany Superabsorbent Polyacrylate Materials from Arkent Ionia, Missouri, or Aquasorb
Silica—SIPERNAT® 50 S from Evonik Operations GmbH, Hanau-Wolfgang, Germany Mineral spirits and mineral oil from Sunnyside Corporation, Wheeling, Illinois POLOX™ polyethylene oxide from DuPont, Wilmington, Delaware
PEG 600—Polyethylene glycol from Dow Chemical, Midland, Michigan SPAN™ 80 and TWEEN™ 60 surfactants from Croda, Snaith, United Kingdom

### Equipment:

Blender—Ross Planetary blender Model 3943, ITW Food Equipment Group, LLC, Troy, Ohio
Fluid pump—Model 515AN3 by Neptune, Toronto, Canada
Volumetric Screw feeder—Model #K2MVS60, by K-Tron Corp., Sewell, New Jersey
Twin Screw Extruder—Model ZSK-30 made by Coperion Corporation, Ramsey, New Jersey Cast Film Take-up and MDO stretch made by Killion Extrduers, a Davis Standard Company, of Pawcatuck, Connecticut

To demonstrate the effect of swelling agent treatment on the durometer or compressibility of a superabsorbent polymer, the force required to compress samples of superabsorbent polymer powder swelled to different extents was tested. More specifically, in this experiment, different samples of superabsorbent polymer powder were placed in a 65 mm diameter cylindrical container to a height of 5 mm. A 60 mm diameter circular plunger was placed on top of the sample of superabsorbent polymer powder, and the superabsorbent polymer powder was compressed by 10%, i.e., from a height of 5.0 mm to a height of 4.5 mm. The force required for this compression is listed below. The three samples tested were: (1) a dry (i.e., as-is) superabsorbent polymer powder, (2) a 200% water-absorbed superabsorbent polymer powder, and (3) a 500% water-absorbed superabsorbent polymer powder. As can be seen, following swelling agent treatment, the force needed to mill the superabsorbent polymer powder was reduced by 30% (for the 200% water-absorbed sample) and by 62% (for the 500% water-absorbed sample).

| | Initial | Compressed | Compression | Compression | Force |
|---|---|---|---|---|---|
| | Ht | Ht to | Force | Pressure | Reduction, |
| Compression of SAP | (5 mm) | 4.5 mm | (kgf) | (g/cm²) | % |
| SAP (Arkent) dry as-is. | 5 | 4.5 | 11.6 | 410 | n/a |
| SAP + 200% water, | 5 | 4.5 | 8.1 | 286 | −30% |
| SAP + 500% water, | 5 | 4.5 | 3.1 | 110 | −62% |

Compression dia. = 60 mm dia. circle
Compression circle area = 28.2744 cm²

In the samples discussed below in Tables 1 and 2, the superabsorbent polymer was first mixed with a swelling agent, which was in the form of water. In some cases, the swelling agent was pre-mixed with an infused agent. The superabsorbent polymer was allowed to swell, thereby reducing the force needed for its compression. Subsequently, the pre-mixed composition was fed to an extruder, the extruder applying shear force to wet-mill the swelled superabsorbent polymer particles, homogenizing the mixture, and forming an extrudate via an extrusion die. The melt was then cast and cooled into a final sheet product.

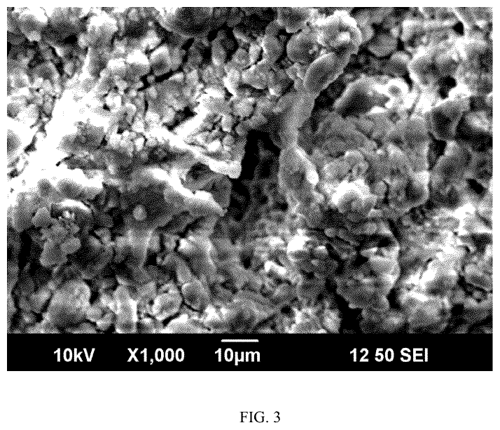

Sample A, which represents an alkaline or lead acid separator, comprises a superabsorbent polymer mixture that was coated onto a nonwoven. Subsequently, the swelling agent in the coating was evaporated at 90° C., thereby producing a separator with 70% porosity. FIG. 3 is a scanning electron microscope (SEM) image of this sample. Water occupies a large portion of the wet volume of this sample, forming large open pores after drying. The final product showed very high air-permeability, i.e., 8 seconds of Gurley number.

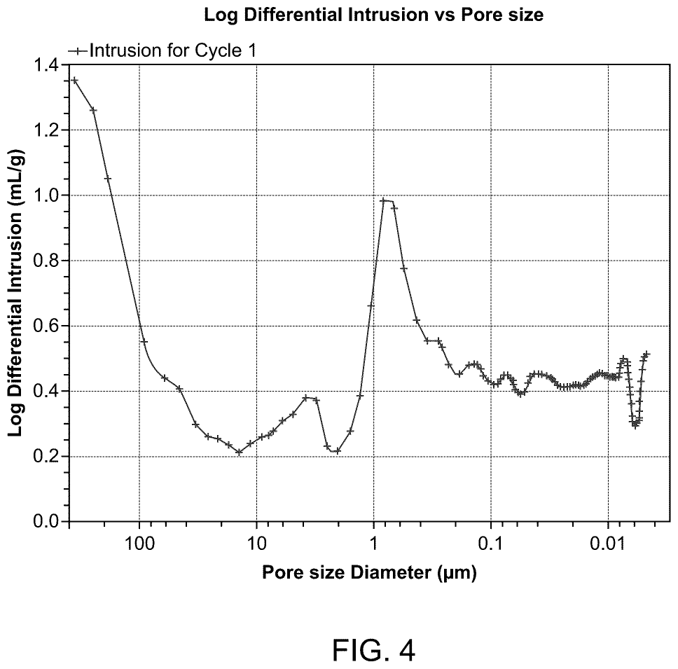

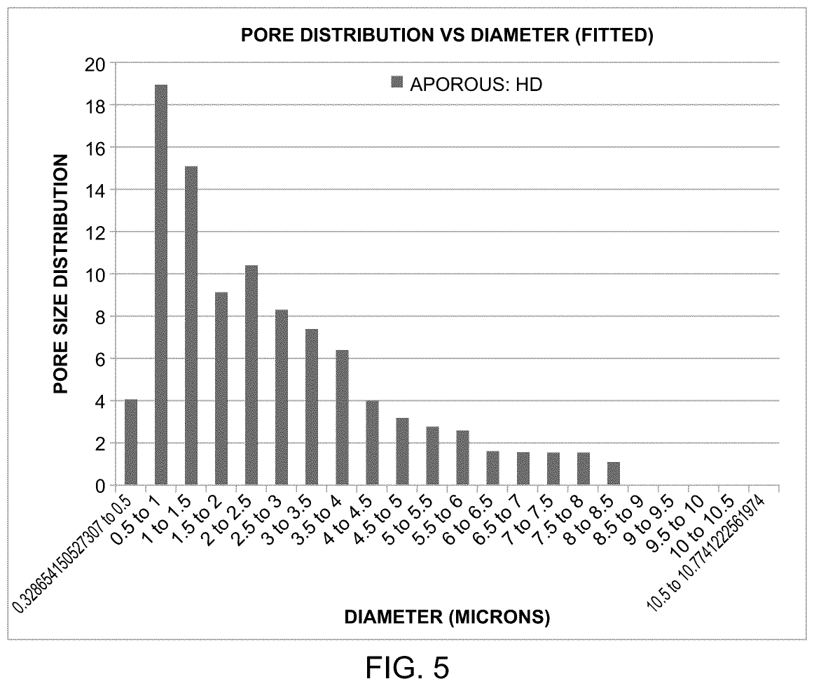

FIG. 4 graphically depicts the pore sizes of Sample A as measured by the mercury porosimetry method. The mercury porosimetry method, which accounts for all pores and volume regardless of shape and tortuosity, shows a bi-modal pore size distribution in this case. As can be seen, the larger mean pore size is about 200 microns whereas the smaller mean pore size is about 1 micron. FIG. 5 graphically depicts the pore sizes of Sample A by the bubble point method, which measures through pores from gas flow. The bubble point porosimetry technique shows a bottleneck effect, accounting pore size by the smallest diameter of the through pores from gas flow. As can be seen, the measured pore size is in the range of 0.3 micron to 8.5 micron, the mode pore size is in the range of 0.5 micron to 1 micron, more specifically about 0.8 micron, and the mean pore size is about 2 microns.

Sample B, which represents a ceramic coated lithium battery separator, was coated by a water-in-oil two-phase solution. The oil phase used cyclic olefin and polyisobutylene as binders dissolved in mineral spirits, the water phase had water-swelled superabsorbent polymer particles, and the substrate was a polyethylene separator.

Sample C represents a coated lithium battery separator. The coating solution was a single-phase homogeneous water solution, with water as the swelling agent for the superabsorbent polymer and with a polyvinyl alcohol dissolved in free water as the polymer binder. The superabsorbent polymer was prepared by first adding water to the superabsorbent polymer, allowing the superabsorbent polymer to swell and to soften, then wet-milling the superabsorbent polymer in an extruder, reducing the superabsorbent polymer particles from about 200 microns down to a size of about 0.1 micron to 1 micron. Subsequently, the wet-milled superabsorbent polymer and silica powder were added to a polyvinyl alcohol solution, forming a homogeneous hydrophilic solution. The foregoing hydrophilic solution was then coated onto a separator by the doctor blade method, with both surfaces coated sequentially. The water was then removed by drying, forming a three-layer sheet product. This is a so-called ceramic coating on a lithium separator. Due to the fact that the above-described sheet product comprises water-swelled superabsorbent polymer particles, when water is removed, porosity is formed by the shrinking of the superabsorbent particles, resulting in a separator exhibiting low ionic resistivity or high lithium ion conductivity. The resistivity of this separator is 380 ohm-cm, which is 50% less than a conventional polyolefin separator.

Sample D represents a food packaging film that uses ethylene-vinyl acetate and polyisobutylene as the thermoplastic polymer, water as the swelling agent, and caramel as the infused agent. Both the swelling agent and the infused agent were absorbed into the superabsorbent polymer. The extrusion mixture was melt-extruded, the superabsorbent polymer particle size was reduced, then water was partially removed by an extruder vent; subsequently, the melt-extrudate was collected. The melt-extrudate was coated onto a nonwoven, forming a final sheet product. When this sheet product contacted a wet paper towel, the caramel color transferred from the sheet product to the paper towel. More specifically, the sheet product color changed from 29 to 44, and the paper towel color changed from 73 to 30, i.e., a color change of −43 points for the paper towel. Color was measured by the L value of a colorimeter, with a lower value representing a darker color.

Sample E represents a food packaging polyamide film, wherein water was used as the swelling agent and wherein glycerol and caramel powder were used as the infused agents. Both the swelling agent and the infused agents were absorbed into the superabsorbent polymer particles, and the mixture was then wet-milled via extrusion. The extrusion reduced the particle size of the superabsorbent polymer particles, the swelling agent was removed by vacuum during extrusion, and glycerin and caramel remained within the superabsorbent polymer. The resultant mixture was subsequently hot-pressed into a polyamide sheet. The above-described treated polyamide sheet product was able to desorb caramel color. More specifically, when exposed to a wet paper towel, the sheet product changed color from 33 to 56, and the contacting paper towel changed color from 69 to 40, i.e., a color change of −29 points for the paper towel.

TABLE 1
Sample Formulation

| Formula and Sample ID | A | B | C | D | E |
|---|---|---|---|---|---|
| SAP | 6% | 1% | 1% | 5% | 5% |
| Water | 41% | 43% | 85% | 44% | 44% |
| cyclic olefin | 0% | 4% | 0% | 0% | 0% |
| polyisobutylene | 10% | 1% | 0% | 4% | 4% |
| Polyamide 612 | 0% | 0% | 0% | 0% | 16% |
| EVA | 0% | 0% | 0% | 1% | 1% |
| Vista | 9% | 1% | 0% | 16% | 0% |
| EVOH | 0% | 0% | 6% | 0% | 0% |
| Silica | 3% | 10% | 8% | 5% | 5% |
| caramel | 0% | 0% | 0% | 5% | 5% |
| Polyethylene glycol | 0% | 0% | 0% | 0% | 8% |
| surfactant* | 0% | 0% | 0% | 2% | 12% |
| mineral oil | 0% | 0% | 0% | 18% | 0% |
| mineral spirits | 31% | 40% | 0% | 0% | 0% |
| Total | 100% | 100% | 100% | 100% | 100% |

*1% polysorbate
*polysorbate
*polysorbate:glycerine (1:2)

TABLE 2
Sample Physical Properties and Performance

| Sample ID | A | B | C | D | E |
|---|---|---|---|---|---|
| Coating Substrate | nonwoven | PE separator | PE separator | nonwoven | none |
| Dry Thickness (micron) | 417 | 641 | 42 | 599 | 320 |
| Wet Thickness (24 hr, micron) | 1074 | 639 | 42 | 896 | 368 |
| Dry Basis Wt (gm/m · sq.) | 272 | 217 | 21 | 663 | 338 |
| IPA Wetout (sec) | instant | 2 | 1 | none | none |
| Porosity (%) | 70% | 63% | 58% | 16% | 9% |
| Water absorption (%, 10 min.) | 234% | 169% | 138% | 19% | 13% |
| Water absorption (%, 24 hr) | 326% | 187% | 142% | 54% | 9% |
| Gurley (sec. per 100 cc) | 8 | 383 | 420 | no flow | no flow |
| Electrolyte type | 30% KOH | Lithium | Lithium | 30% KOH | 30% KOH |
| Resistivity (ohm-cm) | 13 | 1800 | 380 | 112 | 56 |
| Original Caramel Sheet Color (L*) | | | | 29 | 33 |
| Sheet Color (after desorbed, L) | | | | 44 | 56 |
| Delta L from Caramel Desorb | NA | NA | NA | 15 | 23 |
| Original Paper Towel Color (L) | | | | 73 | 69 |
| Paper Towel Absorbed (24 hr, L) | | | | 30 | 40 |
| Absorbed Caramel Color (Delta L) | NA | NA | NA | −43 | −29 |

*colorimeter L range is 100 to 0 (lower number is darker, i.e. 0 is black color).

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

1. A porous sheet product made by a method comprising (i) providing an extrusion mixture, the extrusion mixture comprising a polymeric binder and a superabsorbent polymer, the superabsorbent polymer being in particle form and having a particle size; (ii) milling the superabsorbent polymer such that the particle size of the superabsorbent polymer, before and after milling, is reduced by a ratio of at least 5:1, (iii) melt-extruding the extrusion mixture to form a sheet material in film form; and (iv) cooling the sheet material.

2. The porous sheet product as claimed in claim 1 wherein the milling step is performed before the melt-extruding step.

3. The porous sheet product as claimed in claim 1 wherein the milling step is performed concurrently with the melt-extruding step.

4. The porous sheet product as claimed in claim 1 wherein the milling step is performed while the superabsorbent polymer is present in the extrusion mixture.

5. The porous sheet product as claimed in claim 1 wherein the milling step is performed prior to providing the extrusion mixture.

6. The porous sheet product as claimed in claim 1 wherein the polymeric binder comprises a thermoplastic polymer, the thermoplastic polymer forming an open-celled matrix in the porous sheet product.

7. The porous sheet product as claimed in claim 6 wherein the thermoplastic polymer comprises at least one member selected from the group consisting of polyolefins, fluoropolymers, polyamides, polyethylene terephthalate, polyacrylics, polyvinyl acetate, polyvinyl alcohol, cellulosics, hydroxypropyl cellulose, polyacrylonitrile, polystyrene, polyurethane, polycarbonate, polysulfone, and polyimide.

8. The porous sheet product as claimed in claim 6 wherein the thermoplastic polymer comprises at least one member selected from the group consisting of elastomers of polyamide, polyolefin, polyether, polyisobutylene, polybutadiene, polystyrene, atactic polypropylene, polyurethane, ethylene-propylene rubber, ethylene-vinyl acetate copolymer, polyvinyl chloride, oxidized polyethylene, coumarone-indene resins, and terpene resins.

9. The porous sheet product as claimed in claim 6 wherein the thermoplastic polymer constitutes about 1-80% by volume of the extrusion mixture.

10. The porous sheet product as claimed in claim 1 wherein the superabsorbent polymer comprises at least one member selected from the group consisting of cross-linked polyacrylates, acrylic acid polymers, methacrylates, polyacrylamides, carboxymethyl celluloses, polyvinyl alcohol copolymers, polyethylene oxides, cellulose, starch-grafted copolyacrylates or polyacrylamides, ethylene maleic anhydride copolymers, and copolymers thereof.

11. The porous sheet product as claimed in claim 1 wherein the superabsorbent polymer has an initial, un-milled, dry size of about 20 microns to 5 mm.

12. The porous sheet product as claimed in claim 1 wherein the superabsorbent polymer, prior to swelling or milling, is added to the extrusion mixture and constitutes about 0.1-40% by volume of the extrusion mixture.

13. The porous sheet product as claimed in claim 1 wherein the milling step comprises wet-milling the superabsorbent polymer.

14. The porous sheet product as claimed in claim 13 wherein the method further comprises, prior to the milling step, swelling the superabsorbent polymer.

15. The porous sheet product as claimed in claim 14 wherein the superabsorbent polymer, after swelling, is at least twice as large in any of length, width, and height dimensions as the superabsorbent polymer, prior to swelling.

16. The porous sheet product as claimed in claim 14 wherein the swelling step comprises exposing the superabsorbent polymer to a swelling agent, whereby the superabsorbent polymer absorbs at least a portion of the swelling agent.

17. The porous sheet product as claimed in claim 16 wherein the swelling agent comprises at least one of water and a C1 to C3 alcohol or diol.

18. The porous sheet product as claimed in claim 16 wherein the method further comprises, after forming the sheet material, removing at least a portion of the swelling agent from the sheet material.

19. The porous sheet product as claimed in claim 16 wherein the swelling step further comprises exposing the superabsorbent polymer to an infused agent, whereby the superabsorbent polymer absorbs at least a portion of the infused agent.

20. The porous sheet product as claimed in claim 19 wherein the infused agent is at least one member selected from the group consisting of humectants, antistatic additives, ionic conductivity additives, color additives, flavorings, medications, and indicators.

21. The porous sheet product as claimed in claim 19 wherein the infused agent is present in an amount constituting about 0.1 to 60% by volume of the porous sheet product.

22. The porous sheet product as claimed in claim 19 wherein the method further comprises, after forming the sheet material, removing at least a portion of the swelling agent and/or the infused agent from the sheet material.

23. The porous sheet product as claimed in claim 1 wherein the extrusion mixture further comprises a compatibilizing agent that promotes uniform mixing of the polymeric binder and the superabsorbent polymer at extrusion temperature.

24. The porous sheet product as claimed in claim 23 wherein the compatibilizing agent constitutes about 1-80% by volume of the extrusion mixture.

25. The porous sheet product as claimed in claim 23 wherein the method further comprises, after forming the sheet material, removing at least a portion of the compatibilizing agent from the sheet material.

26. The porous sheet product as claimed in claim 1 wherein the extrusion mixture further comprises an inorganic oxide.

27. The porous sheet product as claimed in claim 1 wherein the porous sheet product consists of a single layer.

28. The porous sheet product as claimed in claim 1 wherein the porous sheet product comprises a plurality of layers.

29. The porous sheet product as claimed in claim 1 wherein the porous sheet product has a thickness of about 0.1-80 mils.

30. A method of producing a porous sheet product, the method comprising the steps of:
(a) providing an extrusion mixture, the extrusion mixture comprising a polymeric binder and a superabsorbent polymer, the superabsorbent polymer being in particle form and having a particle size;
(b) milling the superabsorbent polymer such that the particle size of the superabsorbent polymer before and after milling is reduced by a ratio of at least 5:1,
(c) melt-extruding the extrusion mixture to form a sheet material in film form; and
(d) cooling the sheet material.

31. The method as claimed in claim 30 wherein the milling step is performed before the melt-extruding step.

32. The method as claimed in claim 30 wherein the milling step is performed concurrently with the melt-extruding step.

33. The method as claimed in claim 30 wherein the milling step is performed while the superabsorbent polymer is present in the extrusion mixture.

34. The method as claimed in claim 30 wherein the milling step is performed prior to providing the extrusion mixture.

35. The method as claimed in claim 30 wherein the milling step is a wet-milling step, the method further comprising swelling the superabsorbent polymer with a liquid swelling agent.

36. (canceled)

37. A porous sheet product made by a method comprising (i) wet-milling a first mixture, the first mixture comprising a superabsorbent polymer, a swelling agent, and an infused agent, wherein the superabsorbent polymer is in particle form and has interstices, wherein the swelling agent and the infused agent form a one-phase homogeneous solution that is absorbed into the interstices of the superabsorbent polymer, wherein the wet-milling is such that the particle size of the superabsorbent polymer, before and after wet-milling, is reduced by a ratio of at least 5:1; (ii) melt-extruding a second mixture to form a sheet material in film form, the second mixture comprising the first mixture and a polymeric binder; and (iii) cooling the sheet material.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)
